(19) United States
(12) Patent Application Publication
(10) Pub. No.: US 2025/0143613 A1
(43) Pub. Date: May 8, 2025

(54) Method for Determining Meditators of the Association between Adverse Childhood Experiences and Suicidal Ideation in Late Adulthood and Computing Device for Implementing the Method

(71) Applicant: UNIVERSITY INDUSTRY FOUNDATION, YONSEI UNIVERSITY WONJU CAMPUS, Wonju-si (KR)

(72) Inventors: Moo Kwon CHUNG, Seoul (KR); Kyoung Joung LEE, Wonju-si (KR); Taek Soo SHIN, Wonju-si (KR); Hyo Sang LIM, Wonju-si (KR); Min Hyuk KIM, Wonju-si (KR); Jin Hee LEE, Wonju-si (KR); Sang Won HWANG, Seoul (KR); ERDENEBAYAR URTNASAN, Wonju-si (KR); Jin Kyung LEE, Wonju-si (KR); Ji Young PARK, Wonju-si (KR)

(73) Assignee: UNIVERSITY INDUSTRY FOUNDATION, YONSEI UNIVERSITY WONJU CAMPUS, Wonju-si (KR)

(21) Appl. No.: 18/935,073

(22) Filed: Nov. 1, 2024

(30) Foreign Application Priority Data
Nov. 3, 2023 (KR) 10-2023-0150974

(51) Int. Cl. A61B 5/16; A61B 5/00; G16H 10/20; G16H 10/60

(52) U.S. Cl. CPC A61B 5/165; A61B 5/4845; G16H 10/20; G16H 10/60

(57) ABSTRACT

A method for determining mediators on the association between adverse childhood experiences and suicidal ideation of older adults according to an embodiment of the present disclosure, which is a method for determining mediators on the association between adverse childhood experiences of older adults, as an independent variable, and suicidal ideation, as a dependent variable, may include a data measurement step of measuring data for adverse childhood experiences, suicidal ideation, mental health mediators, physical health mediators, social relationship mediators, and covariates; and a statistical analysis step of estimating adjusted effects and crude effects of the mental health mediators, physical health mediators, and social relationship mediators on the suicidal ideation using a logistic-regression analysis and analyzing three path models respectively using mental health mediators, physical health mediators, and social relationship mediators, which are sets of multiple mediators.

### FIELD OF TECHNOLOGY

The present disclosure relates to a method for determining mediators for relationship between adverse childhood experiences and suicidal ideation of older adults and a computing device for implementing the method.

### BACKGROUND ART

According to the World Health Organization (WHO), seven hundred thousand people over the world pass away by suicide every year and the suicides of older adults are more frequent compared with other age groups. The Republic of Korea (ROK), in particular, is one of the countries in Organization for Economic Cooperation and Development (OECD) having high suicide rates. The suicide rate of older adults in the ROK is 54.8 for 100,000 people, which is quite high, whereas the average suicide rate of the OECD countries is 18.4. As the population ratio of older adults increases, the suicides of older adults become a serious social issue.

Since the probability that older adults have suicidal ideation (SI) can be high compared with people in the other age groups, identifying risk factors and underlying mechanisms can be highly important in developing prevention strategies for suicides of older adults in the ROK.

Recent meta-analyzed research shows that adverse childhood experiences (ACE) are related to suicidal ideation such as suicidal thoughts, attempts, behaviors, etc. According to the interpersonal relations theory, people having adverse childhood experiences (ACE) may feel burdened regarding their parents and have difficulties in feeling a sense of belonging in their earlier stage of development.

Although most conventional research about adverse childhood experiences and suicidal ideation mainly focused on adolescents and young adults, research about the relationship between adverse childhood experiences and suicidal ideation across different age groups has been increasing recently. Since there is a considerable time difference between the exposure to the adverse childhood experiences and the suicide in the senescent period and the adverse childhood experiences affect development processes throughout the life cycle of a person, it is necessary to determine which mediators play important roles in the association between the adverse childhood experiences and the suicidal ideation.

### CONTENTS OF INVENTION

### Problems to be Solved

In this background, in an aspect, the present disclosure is to provide a method for determining mediators on the association between adverse childhood experiences and suicidal ideation of older adults, which is configured to analyze the association between adverse childhood experiences and suicidal ideation of older adults using measured adverse childhood experiences, suicidal ideation, mental health mediators, physical health mediators, social relationship mediators, and covariates, and a computing device for implementing the method.

In another aspect, the present disclosure is to provide a method for determining mediators on the association between adverse childhood experiences and suicidal ideation of older adults, which is configured to estimate adjusted effects and crude effects of mental health mediators, physical health mediators, social relationship mediators on suicidal ideation using a logistic-regression analysis as a preliminary analysis, and a computing device for implementing the method.

Yet in another aspect, the present disclosure is to provide a method for determining mediators on the association between adverse childhood experiences and suicidal ideation of older adults, which is configured to analyze three path models respectively using mental health mediators, physical health mediators, and social relationship mediators, and a computing device for implementing the method.

Technological tasks of the present disclosure are not limited to those mentioned above. Other technological tasks, that are not mentioned above, should be more clearly understood by a person having ordinary skill in the art to which the present disclosure pertains from the following descriptions.

### Means for Solving Problems

A method for determining mediators on the association between adverse childhood experiences and suicidal ideation of older adults according to an embodiment of the present disclosure in order to solve the aforementioned technological problems, which is a method for determining mediators on the association between adverse childhood experiences of older adults, as an independent variable, and suicidal ideation, as a dependent variable, includes a data measurement step of collecting data on adverse childhood experiences, suicidal ideation, mental health mediators, physical health mediators, social relationship mediators, and covariates; and a statistical analysis step of estimating adjusted effects and crude effects of the mental health mediators, physical health mediators, and social relationship mediators on the suicidal ideation using a logistic-regression analysis and analyzing three path models respectively using the mental health mediators, physical health mediators, and social relationship mediators, which are sets of multiple mediators.

According to an embodiment, in the data measurement step, severity of the adverse childhood experiences may be measured using a plurality of binary items of an early trauma inventory (ETI).

According to an embodiment, in the data measurement step, the suicidal ideation may be measured using items of Patient Health Questionnaire-9 (PHQ-9).

According to an embodiment, the mental health mediators may include depression, anxiety, and binge drinking.

According to an embodiment, in the data measurement step, the depression may be measured using items of the Patient Health Questionnaire-9 (PHQ-9), the anxiety may be measured using General Anxiety Disorder-7 (GAD-7), and the binge drinking may be measured by checking if a respondent consumes 5 glasses or more in a single occasion.

According to an embodiment, the physical health mediators may include high blood pressure (HBP), hyperlipidemia (HLP), diabetes mellitus (DM), cardiovascular diseases (CVD), cerebrovascular accidents (CVA), or cancers (CA).

According to an embodiment, the physical health mediators may be measured by coding whether a respondent has ever been diagnosed by a doctor into binary variables.

According to an embodiment, the social relationship mediators may include social supports and social networks.

According to an embodiment, the social supports may be measured by an average of scores for a plurality of items of the Multidimensional Scale of Perceived Social Support (MSPSS) and the social networks may be measured by combining scores for a plurality of items of the Lubben Social Network Scale (LSNS).

According to an embodiment, the covariates may include ages, sexes, levels of education, and household incomes of participants.

According to an embodiment, the crude effects may be to estimate a direct impact of the adverse childhood experiences on the suicidal ideation and the adjusted effect may be to estimate an indirect impact of the adverse childhood experiences on the suicidal ideation through the mental health mediators, physical health mediators, and social relationship mediators.

According to an embodiment, the statistical analysis step may include an analysis step of a path model using mental health mediators and, in the analysis step of a path model using mental health mediators, it may be revealed that the adverse childhood experiences have a positive association with the depression and the anxiety, the higher degrees of the depression and the anxiety are associated with the higher suicidal ideation, and the adverse childhood experiences have an indirect effect on the suicidal ideation through the depression and the anxiety.

According to an embodiment, the statistical analysis step may include an analysis step of a path model using physical health mediators and in the analysis step of a path model using physical health mediators, it may be revealed that those with hyperlipidemia or diabetes mellitus highly likely experience the suicidal ideation and the adverse childhood experiences do not have significant association with the high blood pressure, hyperlipidemia, diabetes mellitus, cardiovascular diseases, cerebrovascular accidents, or cancers.

According to an embodiment, the statistical analysis step may include an analysis step of a path model using social relationship mediators and, in the analysis step of a path model using social relationship mediators, it may be revealed that the adverse childhood experiences have a negative association with the social supports and the social networks, the social supports have a negative association with the suicidal ideation, and the adverse childhood experiences have an indirect effect on the suicidal ideation through the social supports.

A computing device for implementing a method for determining mediators on the association between adverse childhood experiences and suicidal ideation of older adults according to an embodiment of the present disclosure in order to solve the aforementioned technological problems, which is a computing device for implementing a method for determining mediators on the association between adverse childhood experiences of older adults, as an independent variable, and suicidal ideation, as a dependent variable, the computing device comprising at least one processor; a communication interface configured to communicate with external devices; a memory where computer programs are loaded executed by the processor; and a storage device configured to store the computer programs, wherein each computer program may include instructions for estimating adjusted effects and crude effects of mental health mediators, physical health mediators, and social relationship mediators on the suicidal ideation using a logistic-regression analysis and for performing statistical analysis operations of analyzing three path models respectively using mental health mediators, physical health mediators, and social relationship mediators, which are sets of multiple mediators.

According to an embodiment, the mental health mediators may include depression, anxiety, and binge drinking, the physical health mediators may include high blood pressure (HBP), hyperlipidemia (HLP), diabetes mellitus (DM), cardiovascular diseases (CVD), cerebrovascular accidents (CVA), or cancers (CA), and the social relationship mediators may include social supports and social networks.

According to an embodiment, the statistical analysis operations may include analysis operations of a path model using the mental health mediators and, in the analysis operations of a path model using the mental health mediators, it may be revealed that the adverse childhood experiences have a positive association with the depression and the anxiety, the higher degrees of the depression and the anxiety are associated with the higher suicidal ideation, and the adverse childhood experiences have an indirect effect on the suicidal ideation through the depression and the anxiety.

According to an embodiment, the statistical analysis operations may include analysis operations of a path model using the physical health mediators and, in the analysis operations of a path model using the physical health mediators, it may be revealed that those with hyperlipidemia or diabetes mellitus highly likely experience the suicidal ideation and the adverse childhood experiences do not have a significant association with the high blood pressure, hyperlipidemia, diabetes mellitus, cardiovascular diseases, cerebrovascular accidents, or cancers.

According to an embodiment, the statistical analysis operations may include analysis operations of a path model using the social relationship mediators and, in the analysis operations of a path model using the social relationship mediators, it may be revealed that the adverse childhood experiences have a negative association with the social supports and the social networks, the social supports have a negative association with the suicidal ideation, and the adverse childhood experiences have an indirect effect on the suicidal ideation through the social supports.

### Effects of Invention

As described above, according to the present disclosure, a method for determining mediators on the association between adverse childhood experiences and suicidal ideation of older adults, which is configured to analyze the association between adverse childhood experiences and suicidal ideation of older adults using measured adverse childhood experiences, suicidal ideation, mental health mediators, physical health mediators, social relationship mediators, and covariates, and a computing device for implementing the method may be provided.

Additionally, according to the present disclosure, a method for determining mediators on the association between adverse childhood experiences and suicidal ideation of older adults, which is configured to estimate adjusted effects and crude effects of mental health mediators, physical health mediators, social relationship mediators on suicidal ideation using a logistic-regression analysis as a preliminary analysis, and a computing device for implementing the method may be provided.

Further, according to the present disclosure, a method for determining mediators on the association between adverse childhood experiences and suicidal ideation of older adults, which is configured to analyze three path models respectively using mental health mediators, physical health mediators, and social relationship mediators, and a computing device for implementing the method may be provided.

Moreover, various effects understood directly or indirectly by the present specification may be provided.

### BRIEF DESCRIPTION OF THE DRAWINGS

FIG. 1 is a procedure flow diagram of an example to illustrate a method for determining mediators on the association between adverse childhood experiences and suicidal ideation of older adults as an embodiment of the present disclosure.

FIG. 2 is a procedure flow diagram of an example to illustrate in detail a data measurement step of FIG. 1.

FIG. 3 is a procedure flow diagram of an example to illustrate in detail a statistical analysis step of FIG. 1.

FIG. 4 is a procedure flow diagram of an example to illustrate in detail a path model analysis step of FIG. 3.

FIG. 5 is a table showing descriptive statistics of a sample according to an embodiment of the present disclosure.

FIG. 6 is a table showing non-adjusted effects and adjusted effects on suicidal ideation according to an embodiment of the present disclosure.

FIG. 7 is a diagram of path analysis results using mental health mediators according to an embodiment of the present disclosure.

FIG. 8 is a diagram of path analysis results using physical health mediators according to an embodiment of the present disclosure.

FIG. 9 is a diagram of path analysis results using social relationship mediators according to an embodiment of the present disclosure.

FIG. 10 is a block diagram of an example of a computing device for implementing a method for determining mediators on the association between adverse childhood experiences and suicidal ideation of older adults according to an embodiment of the present disclosure.

### DETAILED DESCRIPTION FOR IMPLEMENTING INVENTION

Hereinafter, embodiments of the present disclosure are described in detail with reference to the accompanying drawings. The advantages and features of the present disclosure and methods of achieving the same should be apparent to those having ordinary skill in the art from the following embodiments that are described in detail with reference to the accompanying drawings. It should be noted, however, that the technical spirit of the present disclosure is not limited to the following embodiments. Rather, the technical ideas of the present disclosure may be implemented in different forms. The embodiments described below are provided to fully, thoroughly, and completely convey the technical spirit of the present disclosure to those having ordinary skill in the technical art to which the present disclosure pertains. The technical spirit of the present disclosure is defined by the scope of the claims.

With regard to the reference numerals of the components of the respective drawings, it should be noted that the same reference numerals are assigned to the same components even when the components are shown in different drawings. In addition, in the following description, when it was determined that a detailed description of a related known technology or function may obscure the gist of the present disclosure, the detailed description thereof has been omitted.

If there is no other definition, all the terms (including both technological and scientific terms) used in the present disclosure have meanings that can be commonly understood by persons having ordinary skill in the art to which the present disclosure pertains. In addition, terms shall not be excessively ideally or perfunctorily interpreted if the terms are not clearly and particularly defined as such. The terms used in the present disclosure are only intended to describe embodiments and are not intended to limit the present disclosure. In the present disclosure, a term in a singular form may also mean a term in a plural form as long as there is no particular indication.

In addition, terms, such as “1st”, “2nd”, “A”, “B”, “(a)”, “(b)”, or the like, may be used in describing the components of the present disclosure. These terms are intended only to distinguish a corresponding component from other components, and the nature, order, or sequence of the corresponding component is not limited to the terms. In the case where a component is described as being “coupled”, “combined”, or “connected” to another component, it should be understood that the corresponding component may be directly coupled or connected to another component or that the corresponding component may also be “coupled”, “combined”, or “connected” to the component via another component provided therebetween.

It should be noted that terms, such as ‘comprise’, ‘include’, etc., are intended to indicate the existence of the described characteristics, numbers, steps, operations, components, parts or their combinations. The terms are not intended to preliminarily exclude the existence of other characteristics, numbers, steps, operations, components, parts or their combinations.

Hereinafter, as an embodiment of the present disclosure, a method and a system for determining mediators on the association between adverse childhood experiences and suicidal ideation of older adults are described with reference to the accompanying drawings.

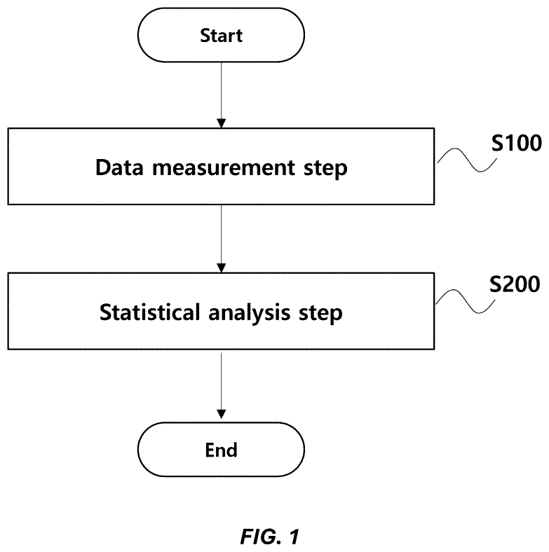

FIG. 1 is a procedure flow diagram of an example to illustrate a method for determining mediators on the association between adverse childhood experiences and suicidal ideation of older adults as an embodiment of the present disclosure.

Referring to FIG. 1, a method for determining mediators on the association between adverse childhood experiences and suicidal ideation of older adults, which is a method for determining mediators on the association between adverse childhood experiences (ACE) as an independent variable and suicidal ideation (SI) as a dependent variable of older adults, as an embodiment of the present disclosure, may comprise a data measurement step (S100) and a statistical analysis step (S200).

The data measurement step (S100) may be a step of measuring data for adverse childhood experiences (ACE), suicidal ideation (SI), mental health mediators, physical health mediators, social relationship mediators, and covariates.

The adverse childhood experiences may be experiences of adverse events or situations including physical, emotional, and sexual abuse, domestic violence, negligence, etc. The adverse childhood experiences (ACE) may cause various adverse problems in mental or physical health, such as suicidal ideation, depression, anxiety, addiction, cardiovascular diseases, chronic diseases.

The suicidal ideation (SI) may refer to thoughts or planning of suicide. The suicidal ideation (SI) is a critical signal that might lead to risks of suicidal attempts or death by suicide. Therefore, it is important to detect it at an early stage so that the person can seek professional assistance.

The mental health mediators, which are factors transferring influences between the adverse childhood experiences (ACE) and the suicidal ideation (SI), may be an intermediate variable related to mental health. For example, the mental health mediators may include depressive symptoms, anxiety, and binge drinking. The mental health mediators may be caused by the adverse childhood experiences (ACE) and affect the suicidal ideation (SI).

The physical health mediators, which are factors transferring influences between the adverse childhood experiences (ACE) and the suicidal ideation (SI), may be an intermediate variable related to physical health. For example, the physical health mediators may include high blood pressure (HBP), hyperlipidemia (HLP), diabetes mellitus (DM), cardiovascular diseases (CVD), cerebrovascular accidents (CVA), and cancers (CA). The physical health mediators may be caused by the adverse childhood experiences (ACE) and affect the suicidal ideation (SI).

The social relationship mediators, which are factors transferring influences between the adverse childhood experiences (ACE) and the suicidal ideation (SI), may be an intermediate variable related to social relationship. For example, the social relationship mediators may include social supports and social networks. The social relationship mediator may be caused by the adverse childhood experiences and affect the suicidal ideation (SI).

The covariates are variables that need to be considered with other variables in a statistical analysis. The covariates are not objects of interest, but are variables that can affect results. For example, the covariates may include, as a variable that can affect association between the adverse childhood experiences (ACE) and the suicidal ideation (SI), ages, sexes, levels of education, and household incomes of participants. By controlling or adjusting the covariates, the association between the adverse childhood experiences (ACE) and the suicidal ideation (SI) may be adjusted or clarified.

The statistical analysis step (S200) may be a step of estimating, as a preliminary analysis, crude effects and adjusted effects of the mental health mediators, physical health mediators, and social relationship mediators on the suicidal ideation (SI) using the logistic-regression analysis and analyzing three path models respectively using mental health mediators, physical health mediators, and social relationship mediators, which are sets of multiple mediators.

The logistic-regression analysis, which is to estimate a probability of occurrence of a dependent variable affected by a linear combination of independent variables, may be used in a case when the dependent variable is a binomial variable (0 or 1, such as occurrence or none). In the logistic-regression analysis, by calculating an odds ratio, a scale of impact of the independent variables on the dependent variable can be identified. The crude effects may be to estimate a direct impact of the adverse childhood experiences on the suicidal ideation and the adjusted effects may be to estimate an indirect impact of the adverse childhood experiences on the suicidal ideation through the mental health mediators, physical health mediators, and social relationship mediators.

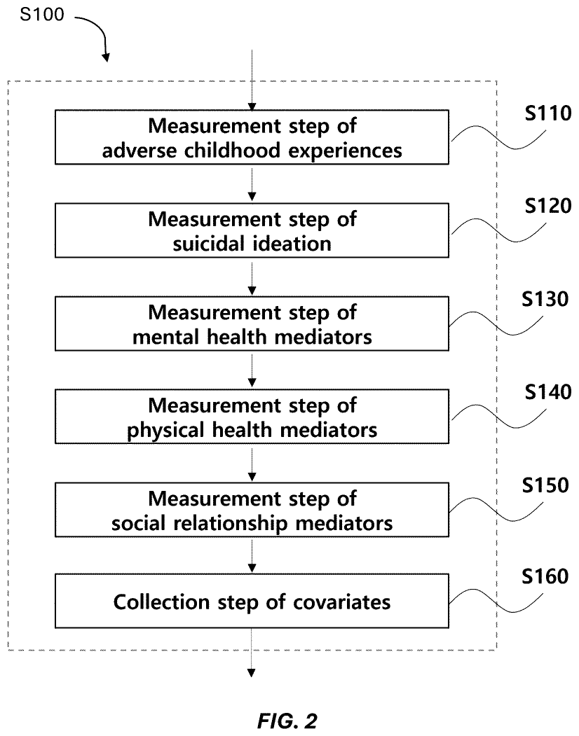

FIG. 2 is a procedure flow diagram of an example to illustrate in detail a data measurement step of FIG. 1.

Referring to FIG. 2, the data measurement step (S100) of FIG. 1 may include a measurement step of adverse childhood experiences (S110), a measurement step of suicidal ideation (S210), a measurement step of mental health mediators (S130), a measurement step of physical health mediators (S140), a measurement step of social relationship mediators (S150), and a collection step of covariates (S160).

The measurement step of adverse childhood experiences (S110) may be a step of measuring severity of adverse childhood experiences using an early trauma inventory (ETI). For example, in measurement step of adverse childhood experiences (S110), adverse childhood experiences may be measured using a shortened version of the early trauma inventory (ETI). Specifically, the severity of adverse childhood experiences may be measured by calculating a combined score using 27 binary items in the early trauma inventory (ETI). The Cronbach's alpha coefficient may be 0.75.

The ETI may be a self-report measurement tool that measures trauma experiences such as physical, emotional, and sexual abuses, accidents, disasters, etc. The ETI may comprise 27 items, for each of which Yes or No responses can be offered regarding whether each experience occurred. The Chonbach's alpha coefficient may be an index indicating internal consistency of scales comprising multiple items. The Chonbach's alpha coefficient may have a value between 0 to 1, where a value closer to 1 indicates a higher reliability. Accordingly, 0.75 of the Cronbach's alpha coefficient refers to a good reliability, which means that the items of the ETI have consistency and are suitable for measuring adverse childhood experiences.

The measurement step of suicidal ideation (S120) may be a step of measuring suicidal ideation (SI) using the Patient Health Questionnaire-9 (PHQ-9).

The PHQ-9, which is a self-report measurement tool for measuring severity of depression symptoms, may comprise 9 items. For each item, how much a respondent suffered from depression symptoms in the last two weeks may be selected among 0 (never) to 3 (almost every day) points. If a total score is 10 or higher out of 27 points, the respondent may be suspected of having depression and if the total score is 15 or higher, it may be determined that the respondent has severe depression.

For example, in the measurement step of suicidal ideation (S120), the severity of suicidal ideation (SI) may be measured using the last item of the PHQ-9. The last item of the PHQ-9 may be a question of how often a respondent has suffered from a thought that it is better for themselves to die or from a thought of hurting themselves in the last two weeks. In the present embodiment, the “0 never” of the original responses may be re-symbolized into “0” and the “1 several days”, “2 over half days”, and “3 almost every day” may all be re-symbolized into “1”.

The measurement step of mental health mediators (S130) may be a step of measuring mental health mediators including depression, anxiety, and binge drinking.

For example, in the measurement step of mental health mediators (S130), the depression may be measured using items (excluding the last item) of the PHQ-9. In the measurement step of mental health mediators (S130), the depression (PHQ-8) may be measured by combining scores of 8 out of 9 items, excluding the item regarding the suicidal ideation. The Cronbach's alpha coefficient may be 0.76. Each item may be answered by scores from “0 never” to “3 almost every day”.

For example, in the measurement step of mental health mediators (S130), degrees of anxiety may be measured using a general anxiety disorder screening tool, the General Anxiety Disorder-7 (GAD-7). The severity of anxiety may be measured by having each item of the GAD-7 answered by scores from “0 never” to “3 almost every day” and calculating a total score for the seven items. The Cronbach's alpha coefficient may be 0.87.

For example, in the measurement step of mental health mediators (S130), binge drinking behavior may be measured. In the measurement step of mental health mediators (S130), the binge drinking may be coded as 1 if a respondent consumes 5 glasses or more in a single occasion and as 0 otherwise in accordance with the definition by the Centers for Disease Control and Prevention (CDC) in the U.S.A.

The measurement step of physical health mediators (S140) may be a step of measuring physical health mediators including high blood pressure (HBP), hyperlipidemia (HLP), diabetes mellitus (DM), cardiovascular diseases (CVD), cerebrovascular accidents (CVA), and cancers (CA).

For example, in the measurement step of physical health mediators (S140), the physical health mediators may be measured by coding whether a respondent has ever been diagnosed by a doctor into binary variables. In the measurement step of physical health mediators (S140), whether a respondent has been diagnosed by a doctor to have high blood pressure (HBP), hyperlipidemia (HLP), diabetes mellitus (DM), cardiovascular diseases (CVD), cerebrovascular accidents (CVA), or cancers (CA) may be coded into binary variables. If the respondent has been diagnosed, it can be coded as (1=Yes), and if not, it can be coded as (0=No).

The measurement step of social relationship mediators (S150) may be a step of measuring social relationship mediators including social supports and social networks. The Cronbach's alpha coefficient for the social relationship mediators measured in the measurement step of social relationship mediators (S150) may be 0.85.

For example, in the measurement step of social relationship mediators (S150), the social supports may be measured by an average of scores for 12 items of the Multidimensional Scale of Perceived Social Support (MSPSS).

For example, in the measurement step of social relationship mediators (S150), the social networks may be measured by combining scores for 18 items of the Lubben Social Network Scale (LSNS).

The collection step of covariates (S160) may be a step of collecting covariates including ages, sexes, levels of education, and household incomes of participants.

For example, in the collection step of covariates (S160), the ages may be ages of participants at a data collection time point. The sexes may be coded into 1 for males and 0 for females. The levels of education, which is a categorical variable, may be coded into 1 for the levels of primary school graduation or below, into 2 for the levels of high school graduation or below, and into 3 for the levels above high school graduation. For the household incomes, average monthly incomes may be indicated by Won, which is the official current unit in Republic of Korea, and a log conversion may be applied in order to adjust a high skewness in the household incomes.

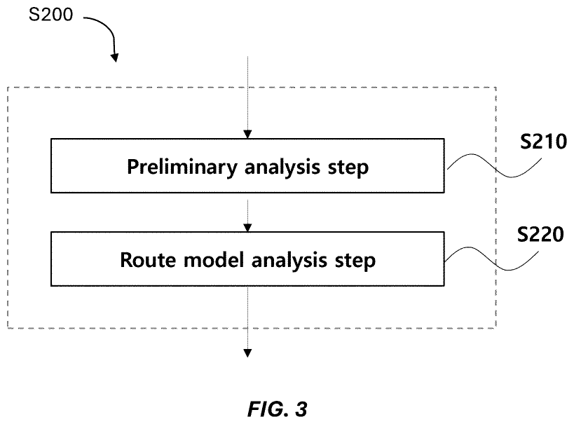

FIG. 3 is a procedure flow diagram of an example to illustrate in detail a statistical analysis step of FIG. 1.

Referring to FIG. 3, the statistical analysis step (S200) may include a preliminary analysis step (S210) and a path model analysis step (S220).

The preliminary analysis step (S210) may be a step of estimating adjusted effects and non-adjusted effects that the mental health mediators, physical health mediators, and social relationship mediators have on the suicidal ideation using the logistic-regression analysis. In the preliminary analysis step (S210), the logistic-regression analysis may be used in order to estimate crude effects and adjusted effects that all the variables have on the suicidal ideation (SI). Here, all the variables may include adverse childhood experiences (ACE), mental health mediators, physical health mediators, social relationship mediators, and covariates.

In an embodiment, a crude effect may be an effect that an independent variable directly has on a dependent variable. For example, a crude effect may be an effect that the adverse childhood experiences have on the suicidal ideation.

In an embodiment, an adjusted effect may be an effect that a mediator, which exists between an independent variable and a dependent variable, has on the independent variable, where the mediator may increase or decrease its effects. For example, when the adverse childhood experiences aggravate the depression and the depression increases the suicidal ideation (SI), the depression may have an adjusted effect between the adverse childhood experiences (ACE) and the suicidal ideation (SI).

The path model analysis step (S220) may be a step of analyzing three path models respectively using mental health mediators, physical health mediators, and social relationship mediators, which are sets of multiple mediators.

For example, a path model may be a model in which causal relationships among independent variables, dependent variables, and mediators are represented by a graph. In a path model, directions and intensities of the causal relationships may be indicated by arrows and variables may be indicated in circles or quadrangles. Using path models may allow easy identifications and analyses of complicated causal relationships.

In other words, in the path model analysis step (S220) after the preliminary analysis step (S210), three path models may be respectively analyzed using three distinct sets of mediators. Covariates such as sex, age, level of education, and household income may be included in predictor variables for estimating the suicidal ideation together with all the mediators. To a moderation model, bootstrapping may be applied repeatedly 5,000 times. As statistical software, STATA 17.0 and Mplus 8.9 may be used.

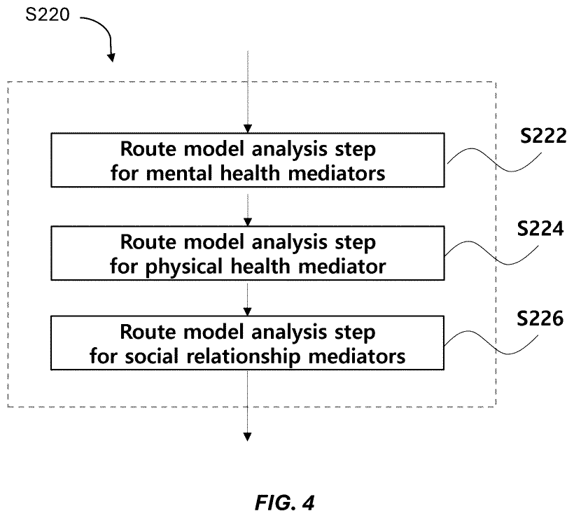

FIG. 4 is a procedure flow diagram of an example to illustrate in detail a path model analysis step of FIG. 3.

Referring to FIG. 4, the path model analysis step (S220) may include a path model analysis step for mental health mediators (S222), a path model analysis step for physical health mediators (S222), and a path model analysis step for social relationship mediators (S222).

The path model analysis step for mental health mediators (S222) may be a step of analyzing association between the suicidal ideation (SI) and the mental health mediators (depression, anxiety, or binge drinking). For example, in the path model analysis step for mental health mediators (S222), direct effects and indirect effects of the mental health mediators on the suicidal ideation (SI) may be measured and a statistically significant result may be derived by controlling covariates (age, sex, level of education, household income).

In an embodiment, the path model analysis step for mental health mediators (S222) may be a step of assessing a path model for the mental health mediators. That is, impacts of mediators related to mental health on the suicidal ideation (SI) and intermediate mediating effects may be identified. For example, how the adverse childhood experiences affect depression and anxiety may be analyzed and impacts of such symptoms of mental health on the suicidal ideation (SI) may also be analyzed.

For example, in the path model analysis step for mental health mediators (S222), it may be revealed that there is a positive association between the adverse childhood experiences and depression and anxiety, higher levels of depression and anxiety are associated with higher levels of the suicidal ideation, and the adverse childhood experiences have an indirect effect on the suicidal ideation through depression and anxiety.

The path model analysis step for physical health mediators (S224) may be a step of analyzing association between the suicidal ideation (SI) and the physical health mediators (high blood pressure, hyperlipidemia, diabetes mellitus, cardiovascular diseases, cerebrovascular accidents, or cancers). For example, in the path model analysis step for physical health mediators (S224), direct effects and indirect effects of the physical health mediators on the suicidal ideation (SI) may be measured and a statistically significant result may be derived by controlling covariates (age, sex, level of education, household income).

In an embodiment, the path model analysis step for physical health mediators (S224) may be a step of assessing a path model for mediators related to the physical health. In this step, how issues of physical health are related to the suicidal ideation (SI) may be identified and a path between the adverse childhood experiences and the physical health mediators may also be identified.

For example, in the path model analysis step for physical health mediators (S224), it may be analyzed that people with hyperlipidemia or diabetes mellitus are highly likely to experience suicidal ideation, and that there is no significant association between the adverse childhood experiences and high blood pressure, hyperlipidemia, diabetes mellitus, cardiovascular diseases, cerebrovascular accidents, or cancers.

The path model analysis step for social relationship mediators (S226) may be a step of analyzing association between the suicidal ideation (SI) and the social relationship mediators (social supports or social networks). For example, in the path model analysis step for social relationship mediators (S226), direct effects and indirect effects of the social relationship mediators on the suicidal ideation (SI) may be measured and a statistically significant result may be derived by controlling covariates (age, sex, level of education, household income).

In an embodiment, the path model analysis step for social relationship mediators (S226) may be a step of assessing a path model for mediators related to the social relationships. For example, in the path model analysis step for social relationship mediators (S226), the association between the adverse childhood experiences (ACE) and the social relationship may be identified and impacts of social supports and social networks on the suicidal ideation (SI) may be examined.

For example, in the path model analysis step for social relationship mediators (S226), it may be revealed that there is a negative association between the adverse childhood experiences and the social supports or the social networks, there is a negative association between the social supports and the suicidal ideation, and there are indirect effects of the adverse childhood experiences on the suicidal ideation through the social supports.

Hereinafter, specific analysis processes for a method for determining mediators on the association between adverse childhood experiences and suicidal ideation for older adults and analysis results are described.

The specific analysis processes for a method for determining mediators on the association between adverse childhood experiences and suicidal ideation for older adults and the analysis results are based on data from one-on-one interviews conducted on campus with Korean elderly individuals aged 55 or older, without serious diseases, who were recruited via telephone calls.

More specifically, the data used for the analysis was collected from 685 Korean elderly individuals. Participants were aged 55 or older and without drug abuse problems, dementia, or serious diseases. For securing data, each interview was conducted for 90 minutes. The participants were recruited through “Korean Genome and Epidemiology Study-Cardiovascular Disease Study”, which is a national cohort study. The participants, who expressed their intentions to participate in a project for studying depression in older adults, visited the campus from December 2020 to April 2021. They were informed of the present analysis process, voluntarily agreed to the process through written consent, and conducted one-on-one interview sessions with trained researchers.

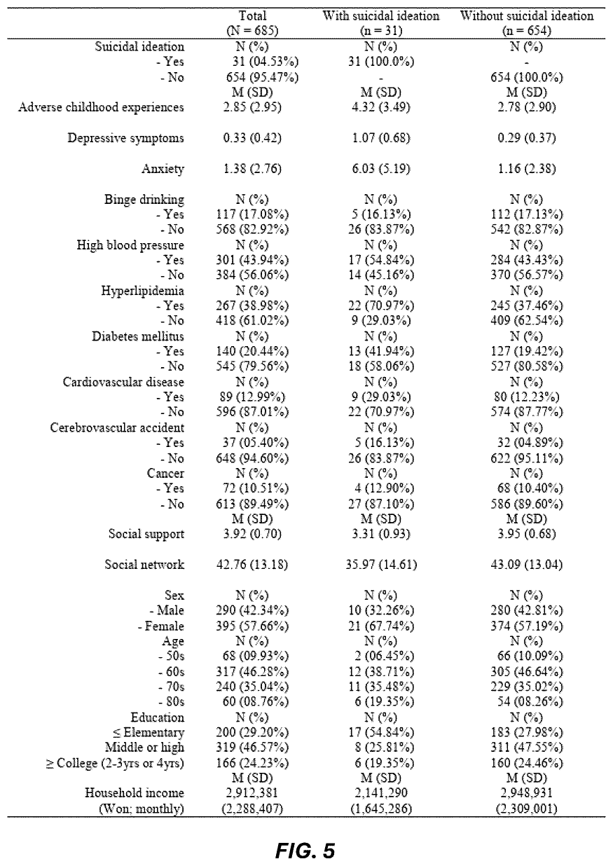

FIG. 5 is a table showing descriptive statistics of a sample according to an embodiment of the present disclosure.

Referring to FIG. 5, 31 (4.53%) among 685 elderly individuals had suicidal ideation (SI) during the previous month. Participants with suicidal ideation (SI) had higher scores for the adverse childhood experiences (ACE), depression symptoms, and anxiety symptoms than participants without suicidal ideation (SI). Older adults individuals with suicidal ideation (SI) had higher occurrence rates of all six health problems than those without suicidal ideation (SI). However, the participants with suicidal ideation (SI) had lower scores for the social supports and social networks than those without suicidal ideation (SI).

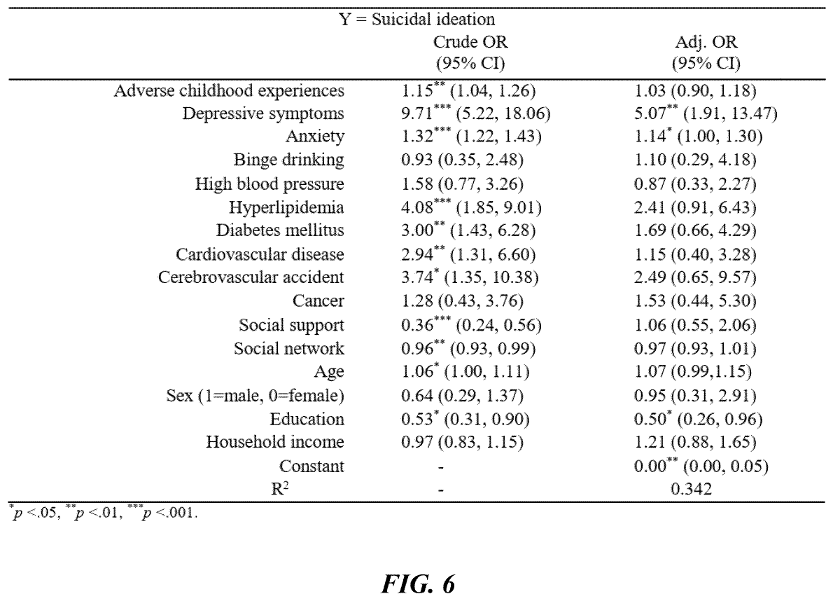

FIG. 6 is a table showing non-adjusted effects and adjusted effects on suicidal ideation according to an embodiment of the present disclosure.

FIG. 6 shows crude effects and adjusted effects of all the variables on the suicidal ideation (SI). Referring to the crude effects, it was revealed that the adverse childhood experiences (ACE) (OR=1.15, p<0.01), depressive symptoms (OR=9.71, p<0.001), anxiety (OR=1.32, p<0.001), high blood pressure (HLP)(OR=4.08, p<0.01), diabetes mellitus (DM) (OR=3.00, p<0.01), cardiovascular diseases (CVD)(OR=2.94, p<0.01), and cerebrovascular accidents (CVA) (OR=3.74, p<0.05) have positive association s with the suicidal ideation (SI). It was also revealed that the social supports (OR=0.36, p<0.001) and social networks ((OR=0.96, p<0.01) have negative association s with the suicidal ideation (SI). However, in an adjusted model in consideration of all the predictor variables, only direct impacts of the depressive symptoms (OR=5.07, p<0.01) and anxiety (OR=1.14, p<0.05) were statistically significant in explaining the suicidal ideation (SI).

Here, the OR, which is an abbreviation of “odds ratio”, may be a statistic indicating the strength of the association between two events A and B, and p, which is an abbreviation of “p-value”, may be an index indicating a statistical significance. For example, the data “adverse childhood experiences (OR=1.15, p<0.01)” may indicate that the adverse childhood experiences (ACE) have a positive association with the suicidal ideation (SI) and this association has a statistical significance at a significance level of 0.01. That is, the higher score of the adverse childhood experiences may be associated with the higher probability of the suicidal ideation (SI).

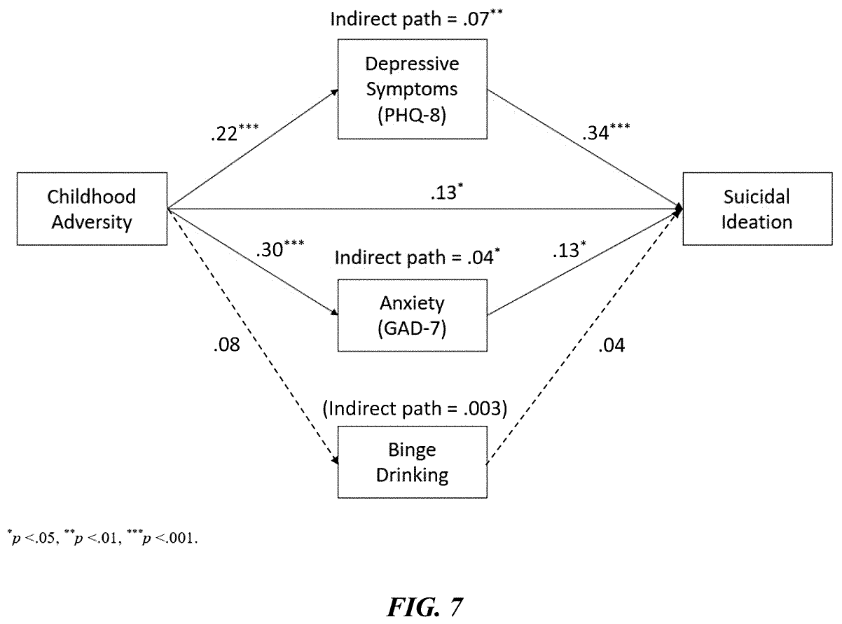

FIG. 7 is a diagram of results of a first path analysis using mental health mediators according to an embodiment of the present disclosure.

Referring to FIG. 7, a path model using mental health mediators may be a saturated model. Its model fix indexes are as follows: χ²(df)=734.74 (26), p=<0.001, RMSEA=0.000, CFI=1.000. A saturated model, which considers all possible relationships between variables, may be a model that fully explains the relationship between given data and the model itself. The χ² (df) is a statistic used for assessing the model fit. The lower value of χ² (df) may indicate the better model fit. The p-value is an index indicating the significance of the value of χ². When the p-value is smaller than 0.05, it may indicate that the model fit is statistically significant. The RMSEA, which is root mean square error of approximation, is an index used for assessing the model fit. The RMSEA value approaching 0 may indicate a better model fit. The CFI is a fit index, which is a value for assessing the model fit by comparing the model with a reference model. The CFI value closer to 1 may indicate a better model fit.

Based on the above indexes, a first path model using mental health mediators has a statistically significant model fit because the p-value is less than 0.001 despite the χ² value of 734.74. Additionally, the first path model demonstrates a very good fit as indicated by the RMSEA value of 0.000 and the CFI value of 1.000.

This model demonstrates that the adverse childhood experiences (ACE) have a positive association with the depressive symptoms (β=22, p<0.001) and the anxiety (β30, p<0.001) and that the higher scores for the depressive symptoms (β=34, p<0.001) and the anxiety (β=13, p<0.05) are associated with the higher suicidal ideation (SI). The adverse childhood experiences (ACE) have a significant indirect effect on the suicidal ideation (SI) through the depressive symptoms (β=0.07, p<0.01) and the anxiety (β=0.04, p<0.05). However, the adverse childhood experiences (ACE) were not significant in predicting binge drinking and binge drinking was not related to the suicidal ideation (SI). Even considering all the mediating routes, a direct effect of the adverse childhood experiences (ACE) was still significant in explaining the suicidal ideation (SI) (β=0.13, p<0.05).

Here, the B, which is a regression coefficient in a regression analysis, may be a value indicating a change amount of a dependent variable when an independent variable increases by 1 unit. For example, the data “depressive symptoms (β=0.22)” may indicate that the depressive symptoms increase by 0.22 units when the adverse childhood experiences (ACE) increase by 1 unit. The p may be a p-value for a regression coefficient in a regression analysis. For example, the data “depressive symptoms (p<0.001)” may indicate that the p-value is less than 0.001. When the p-value is less than 0.05, the association may be considered to be statistically significant. This may be interpreted that there is a significant association between the independent variable “adverse childhood experiences (ACE)” and the variable “depressive symptoms”.

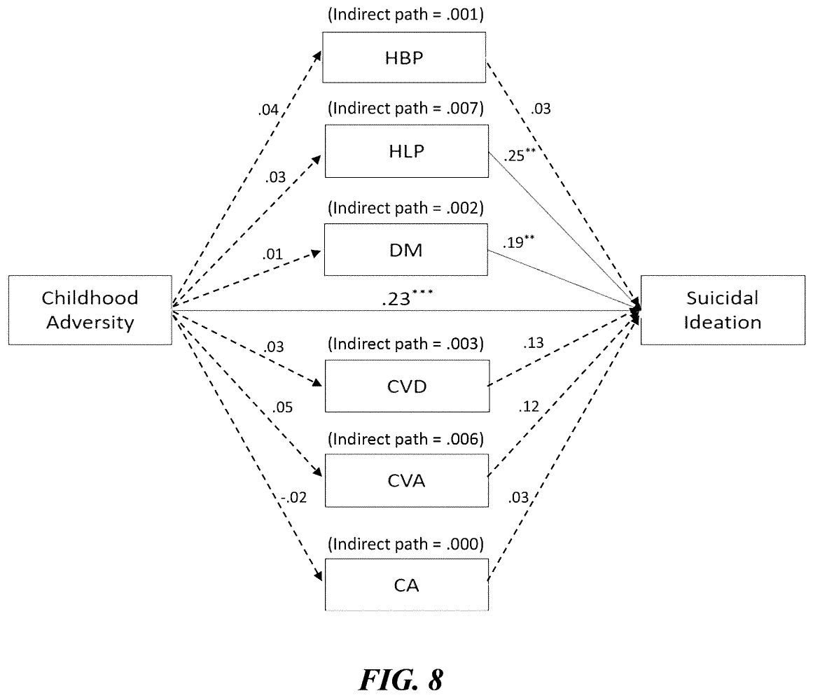

FIG. 8 is a diagram of results of a second path analysis using physical health mediators according to an embodiment of the present disclosure.

Referring to FIG. 8, those with hyperlipidemia (HLP) (β=0.25, p<0.01) and diabetes mellitus (DM) (β=0.19, p<0.01) have high probabilities of experiencing suicidal ideation (SI), but the adverse childhood experiences (ACE) do not have any significant association with physical health problems such as high blood pressure (HBP), hyperlipidemia (HLP), diabetes mellitus (DM), cardiovascular diseases (CVD), cerebrovascular accidents (CVA), and cancers (CA). The model fit indexes are as follows: χ²(df)=93.45 (56), p=0.001, RMSEA=0.027, CFI=0.804.

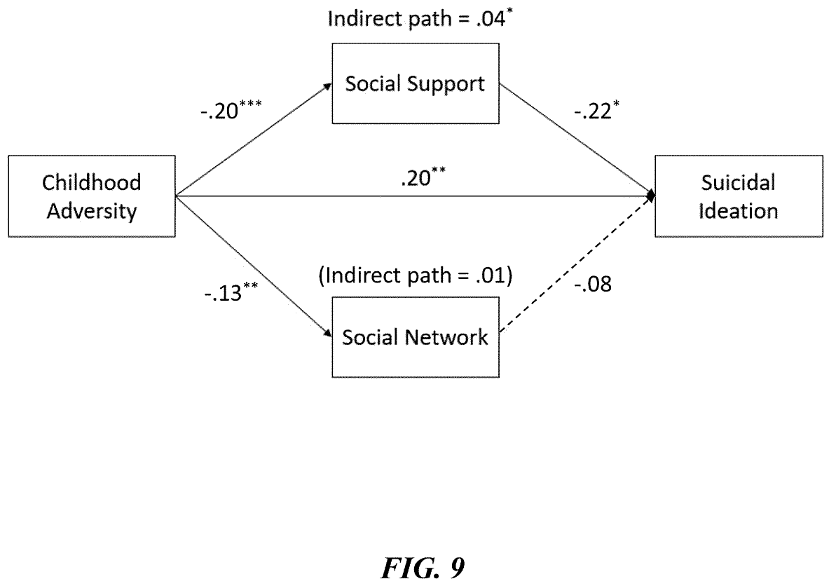

FIG. 9 is a diagram of results of a third path analysis using social relationship mediators according to an embodiment of the present disclosure.

Referring to FIG. 9, model fit indexes of the third path model using social relationship mediators demonstrate that the third path model is a saturated model (χ²(df)=273.58 (18), p<0.001, RMSEA=0.000, CFI=1.000). The adverse childhood experiences (ACE) have negative association s with the social supports (β=−0.20, p<0.001) and the social networks (β=−0.13, p<0.01) and the social supports have a negative association with the suicidal ideation (SI) (β=−0.22, p<0.05). There is a statistically significant indirect effect between the adverse childhood experiences (ACE) and the suicidal ideation (SI) through the social supports (β=0.04, p<0.05). However, the social networks are not significantly related to the suicidal ideation (SI), which demonstrates that the social networks do not mediate a path from the adverse childhood experiences (SI) to the suicidal ideation (SI).

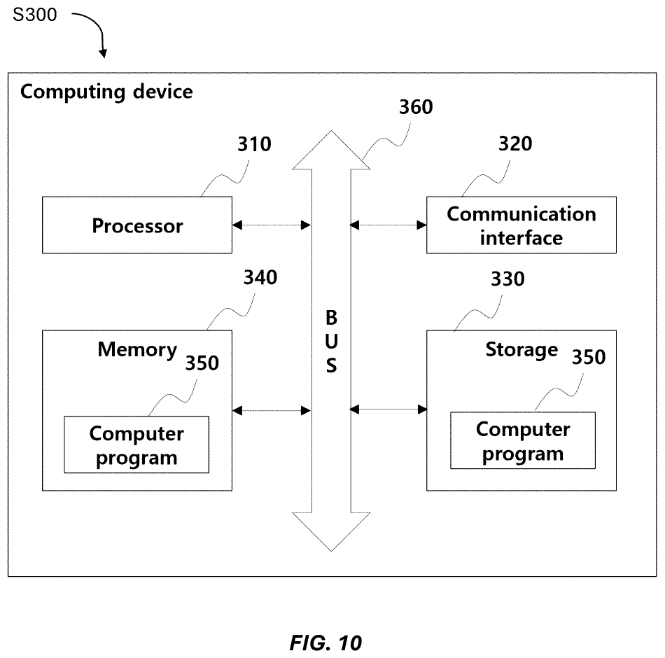

FIG. 10 is a block diagram of an example of a computing device for implementing a method for determining mediators on the association between adverse childhood experiences and suicidal ideation of older adults according to an embodiment of the present disclosure.

FIG. 13 is an example of a block diagram of a computing device for implementing a method for determining mediators on the association between adverse childhood experiences and suicidal ideation of older adults according to an embodiment of the present disclosure.

Referring to FIG. 13, an example of a computing device (300) for implementing a method for determining mediators on the association between adverse childhood experiences and suicidal ideation of older adults according to an embodiment of the present disclosure may comprise a processor (310), a system bus (360), a communication interface (320), a memory (340), and a storage (330).

The processor (310) may control overall operations of the method for determining mediators on the association between adverse childhood experiences and suicidal ideation of older adults. For example, the processor (310) may be a component to estimate adjusted effects and crude effects of mental health mediators, physical health mediators, and social relationship mediators on suicidal ideation using a logistic-regression analysis as a preliminary analysis and to control statistical analysis operations of analyzing three path models respectively using mental health mediators, physical health mediators, and social relationship mediators, which are sets of multiple mediators.

The memory (340) may store various data, commands, and information for executing computer programs (350) and may load at least one computer program (350) from the storage (330) to perform operations according to embodiments of the present disclosure. The bus (360) may provide communication functions among components of the computing device (300). The communication interface (320) may comprise a communication module that supports various communication ways such as wired or wireless internet communications for the computing device (300). The storage (330) may non-temporarily store at least one computer program (350).

Each computer program (350) may include one or more instructions in which operations according to embodiment of the present disclosure are implemented. When a computer program (350) is loaded on the memory (340), the processor (310) may execute one or more instructions to perform operations according to embodiments of the present disclosure.

In an embodiment, each computer program (350) may include an instruction to estimate adjusted effects and crude effects of mental health mediators, physical health mediators, and social relationship mediators on suicidal ideation using a logistic-regression analysis as a preliminary analysis and to perform statistical analysis operations of analyzing three path models respectively using mental health mediators, physical health mediators, and social relationship mediators, which are sets of multiple mediators; an instruction to perform analysis operations of a path model using mental health mediators, which demonstrate an analysis that adverse childhood experiences have a positive association with the depressive symptoms and the anxiety, the higher scores for the depressive symptoms and the anxiety are associated with higher suicidal ideation, and the adverse childhood experiences have a significant indirect effect on the suicidal ideation through the depressive symptoms and the anxiety; an instruction to perform analysis operations of a path model using physical health mediators, which demonstrate an analysis that those with hyperlipidemia and diabetes mellitus have high probabilities of experiencing suicidal ideation, but the adverse childhood experiences do not have any significant association with physical health problems such as high blood pressure, hyperlipidemia, diabetes mellitus, cardiovascular diseases, cerebrovascular accidents, and cancers; and an instruction to perform analysis operations of a path model using social relationship mediators, which demonstrate an analysis that adverse childhood experiences have negative association s with social supports and social networks, the social supports have a negative association with the suicidal ideation, and there is a statistically significant indirect effect between the adverse childhood experiences and the suicidal ideation through the social supports.

As described above, the present disclosure may provide a method for determining mediators on the association between adverse childhood experiences and suicidal ideation of older adults, which is configured to analyze the correlation between adverse childhood experiences and suicidal ideation of older adults using measured adverse childhood experiences, suicidal ideation, mental health mediators, physical health mediators, social relationship mediators, and covariates, and a computing device for implementing the method. Also, the present disclosure may provide a method for determining mediators on the association between adverse childhood experiences and suicidal ideation of older adults, which is configured to estimate adjusted effects and crude effects of mental health mediators, physical health mediators, social relationship mediators on suicidal ideation using a logistic-regression analysis as a preliminary analysis, and a computing device for implementing the method. Further, the present disclosure may provide a method for determining mediators on the association between adverse childhood experiences and suicidal ideation of older adults, which is configured to analyze three path models respectively using mental health mediators, physical health mediators, and social relationship mediators, and a computing device for implementing the method.

In the above, various embodiments of the present disclosure and effects from the embodiments are described with reference to FIG. 1 to FIG. 10. Effect according to technological ideas of the present disclosure are not limited to those mentioned above. Other technological effects, that are not mentioned above, should be more clearly understood by a person having ordinary skill in the art to which the present disclosure pertains from the above descriptions.

Technological ideas of the present disclosed described above may be implemented by computer readable codes on computer readable media. For example, computer readable recording media may be mobile recording media (such as a CD, a DVD, a Blu-ray disk, a USB storage device, a mobile hard disk) or stationary recording media (such as a ROM, a RAM, or a computer fixed hard disk). A computer program recorded on a computer readable recording medium can be transmitted to other computing devices via networks such as the Internet, installed on those devices, and executed on them for use.

Although the steps in the figures are presented in a specific order, they do not necessarily have to be executed in that order or sequentially to achieve the desired results. Not all depicted steps are required to be performed. In certain situations, multitasking and parallel processing may be advantageous. Moreover, in the above-described embodiments, various separations of components should not necessarily be understood as essential. Program components, program modules, and systems may generally be integrated into a single software product or implemented as multiple software packages.

While the embodiments of the present disclosure have been described with reference to the accompanying drawings, it will be understood by those skilled in the art to which the present disclosure pertains that the invention may be embodied in other specific forms without departing from the technical spirit or essential characteristics thereof. Therefore, the above-described embodiments should be understood as illustrative in all respects and not as restrictive. The scope of protection of the present invention should be interpreted based on the following claims, and all technical ideas within the equivalent scope thereof should be construed as falling within the scope of the technical ideas defined by the present disclosure.

What is claimed is:

1. A method for determining mediators on the association between adverse childhood experiences and suicidal ideation of older adults, which is a method for determining mediators on the association between adverse childhood experiences of older adults, as an independent variable, and suicidal ideation, as a dependent variable, including
a data measurement step of collecting data on adverse childhood experiences, suicidal ideation, mental health mediators, physical health mediators, social relationship mediators, and covariates; and
a statistical analysis step of estimating adjusted effects and crude effects of the mental health mediators, physical health mediators, and social relationship mediators on the suicidal ideation using a logistic-regression analysis and analyzing three path models respectively using mental health mediators, physical health mediators, and social relationship mediators, which are sets of multiple mediators.

2. The method for determining mediators on the association between adverse childhood experiences and suicidal ideation of older adults of claim 1, wherein, in the data measurement step, severity of the adverse childhood experiences is measured using a plurality of binary items of the Early Trauma Inventory (ETI).

3. The method for determining mediators on the association between adverse childhood experiences and suicidal ideation of older adults of claim 1, wherein, in the data measurement step, the suicidal ideation is measured using items of Patient Health Questionnaire-9 (PHQ-9).

4. The method for determining mediators on the association between adverse childhood experiences and suicidal ideation of older adults of claim 1, wherein the mental health mediators include depression, anxiety, and binge drinking.

5. The method for determining mediators on the association between adverse childhood experiences and suicidal ideation of older adults of claim 4, wherein, in the data measurement step, the depression is measured using items of the Patient Health Questionnaire-9 (PHQ-9), the anxiety is measured using General Anxiety Disorder-7 (GAD-7), and the binge drinking is measured by checking if a respondent consumes 5 glasses or more in a single occasion.

6. The method for determining mediators on the association between adverse childhood experiences and suicidal ideation of older adults of claim 1, wherein the physical health mediators may include high blood pressure (HBP), hyperlipidemia (HLP), diabetes mellitus (DM), cardiovascular diseases (CVD), cerebrovascular accidents (CVA), or cancers (CA).

7. The method for determining mediators on the association between adverse childhood experiences and suicidal ideation of older adults of claim 6, wherein, in the data measurement step, the physical health mediators are measured by coding whether a respondent has ever been diagnosed by a doctor into binary variables.

8. The method for determining mediators on the association between adverse childhood experiences and suicidal ideation of older adults of claim 1, wherein the social relationship mediators may include social supports and social networks.

9. The method for determining mediators on the association between adverse childhood experiences and suicidal ideation of older adults of claim 8, wherein, in the data measurement step, the social supports are measured by an average of scores for a plurality of items of the Multidimensional Scale of Perceived Social Support (MSPSS) and the social networks are measured by combining scores for a plurality of items of the Lubben Social Network Scale (LSNS).

10. The method for determining mediators on the association between adverse childhood experiences and suicidal ideation of older adults of claim 1, wherein the covariates may include ages, sexes, levels of education, and household incomes of participants.

11. The method for determining mediators on the association between adverse childhood experiences and suicidal ideation of older adults of claim 1, wherein the crude effects are to estimate a direct impact of the adverse childhood experiences on the suicidal ideation and the adjusted effects are to estimate an indirect impact of the adverse childhood experiences on the suicidal ideation through the mental health mediators, physical health mediators, and social relationship mediators.

12. The method for determining mediators on the association between adverse childhood experiences and suicidal ideation of older adults of claim 4, wherein the statistical analysis step includes an analysis step of a path model using mental health mediators and, in the analysis step of a path model using mental health mediators, it is revealed that the adverse childhood experiences have a positive correlation with the depression and the anxiety, the higher degrees of the depression and the anxiety are associated with the higher suicidal ideation, and the adverse childhood experiences have an indirect effect on the suicidal ideation through the depression and the anxiety.

13. The method for determining mediators on the association between adverse childhood experiences and suicidal ideation of older adults of claim 6, wherein the statistical analysis step includes an analysis step of a path model using physical health mediators and, in the analysis step of a path model using physical health mediators, it is revealed that those with hyperlipidemia or diabetes mellitus highly likely experience the suicidal ideation and the adverse childhood experiences do not have significant association with the high blood pressure, hyperlipidemia, diabetes mellitus, cardiovascular diseases, cerebrovascular accidents, or cancers.

14. The method for determining mediators on the association between adverse childhood experiences and suicidal ideation of older adults of claim 9, wherein the statistical analysis step includes an analysis step of a path model using social relationship mediators and, in the analysis step of a path model using social relationship mediators, it is revealed that the adverse childhood experiences have a negative association with the social supports and the social networks, the social supports have a negative association with the suicidal ideation, and the adverse childhood experiences have an indirect effect on the suicidal ideation through the social supports.

15. A computing device for implementing a method for determining mediators on the association between adverse childhood experiences of older adults, which is as an independent variable, and suicidal ideation, as a dependent variable, the computing device comprising,
at least one processor;
a communication interface configured to communicate with external devices;
a memory where computer programs executed by the processor are loaded; and
a storage device configured to store the computer programs,
wherein each computer program includes instructions for estimating adjusted effects and crude effects of mental health mediators, physical health mediators, and social relationship mediators on suicidal ideation using a logistic-regression analysis and for performing statistical analysis operations of analyzing three path models respectively using mental health mediators, physical health mediators, and social relationship mediators, which are sets of multiple mediators.

16. The computing device of claim 15, wherein the mental health mediators include depression, anxiety, and binge drinking, the physical health mediators include high blood pressure (HBP), hyperlipidemia (HLP), diabetes mellitus (DM), cardiovascular diseases (CVD), cerebrovascular accidents (CVA), or cancers (CA), and the social relationship mediators include social supports and social networks.

17. The computing device of claim 16, wherein the statistical analysis operations include analysis operations of a path model using the mental health mediators and, in the analysis step of a path model using social relationship mediators, it is revealed that the adverse childhood experiences have a positive association with the depression and the anxiety, the higher degrees of the depression and the anxiety are associated with the higher suicidal ideation, and the adverse childhood experiences have an indirect effect on the suicidal ideation through the depression and the anxiety.

18. The computing device of claim 16, wherein the statistical analysis operations include analysis operations of a path model using the physical health mediators and, in the analysis operations of a path model using the physical health mediators, it is revealed that those with hyperlipidemia or diabetes mellitus highly likely experience the suicidal ideation and the adverse childhood experiences do not have significant association with the high blood pressure, hyperlipidemia, diabetes mellitus, cardiovascular diseases, cerebrovascular accidents, or cancers.

19. The computing device of claim 16, wherein the statistical analysis operations include analysis operations of a path model using the social relationship mediators and, in the analysis operations of a path model using the social relationship mediators, it is revealed that the adverse childhood experiences have a negative association with the social supports and the social networks, the social supports have a negative association with the suicidal ideation, and the adverse childhood experiences have an indirect effect on the suicidal ideation through the social supports.
